(19) United States
(12) Patent Application Publication
(10) Pub. No.: US 2016/0286820 A1
(43) Pub. Date: Oct. 6, 2016

(54) FERMENTED COCONUT ANTIMICROBIAL PRODUCT

(71) Applicant: Active Micro Technologies, LLC, Lincolnton, NC (US)

(72) Inventors: Durant Scholz, Nanuet, NY (US); Erica Segura, Huntersville, NC (US)

(21) Appl. No.: 15/066,372

(22) Filed: Mar. 10, 2016

Related U.S. Application Data
(60) Provisional application No. 62/139,908, filed on Mar. 30, 2015.

(51) Int. Cl. A01N 63/02; A61Q 19/00; C12P 1/04; A61K 8/97

(52) U.S. Cl. CPC A01N 63/02; A61K 8/97; A61Q 19/00; C12P 1/04; A61K 2800/524; A61K 2800/85

(57) ABSTRACT

An antimicrobial and antifungal composition and method is disclosed. The antimicrobial and antifungal composition is prepared by a process of using Lactobacillus to ferment Cocos Nucifera fruit.

### BACKGROUND OF THE INVENTION

The invention relates to the field of personal care products, and more particularly to antimicrobial compositions for use with personal care products.

Various antimicrobial compositions are well-known in the art. They are used in several industries such as personal care products, water treatment, food applications, and medicine and healthcare. For example, in personal care products, antimicrobials are used to help retard or eliminate the growth of topical pathogens as well as to preserve the personal care products from spoiling or to substantially improve the shelf-life of a product. The antimicrobials can also be used to preserve the function of various active ingredients including anti-oxidants (vitamins), emulsifiers, and surfactants.

Previously-acceptable antimicrobials are now subject to changing government regulations and increasing safety concerns among consumers. The use of antimicrobials such as parabens, formaldehyde donors, and ingredients that dissociate into formaldehyde in aqueous solution is becoming less acceptable.

This situation has lead to a quest for natural antimicrobial products that can suit the needs of formulators and consumers. The challenge is not only finding one product that will protect against a wide range of microorganisms including fungi without adding any synthetic ingredients.

Accordingly, there is a need for natural antimicrobial that is also effective as an antifungal.

### BRIEF SUMMARY OF THE INVENTION

This need is addressed by the present invention, which provides an antimicrobial product developed by fermenting Cocos Nucifera (coconut) fruit with Lactobacillus.

An antimicrobial and antifungal composition for use with personal care products, prepared by a process of macerating white fruit of Cocos Nucifera to create a feedstock material; mixing the feedstock material with a bacteria and using the bacteria to ferment the feedstock material to create a fermented product; and filtering the fermented product to clarify the product and remove remaining feedstock material and produce the composition.

A method of making an antimicrobial and antifungal composition, comprising the steps of macerating white fruit of Cocos Nucifera to produce a feedstock material; mixing the feedstock material with a bacteria to create a pre-fermentation composition and using the bacteria to ferment the feedstock material to create a fermented product; and filtering the fermented product to remove remaining feedstock material and produce the composition.

### DETAILED DESCRIPTION OF THE INVENTION

The present invention provides a natural antifungal product developed by fermenting Cocos Nucifera (coconut) fruit with Lactobacillus to produce a non-irritating product which is effective at preventing the growth of fungus, specifically yeast and mold.

The antifungal product of the current invention is produced using a fermentation process. The production process begins by macerating the white fruit of Cocos Nucifera (coconut), to release liquid therein and expose the surface area of the fruit, thereby producing a feedstock.

The feedstock is then mixed with bacteria, specifically Lactobacillus in a growth culture. The mass of fruit is selected relative to the mass of Lactobacillus to permit fermentation to proceed to completion. In one example the mass of fruit may be about one-quarter the mass of Lactobacillus, also described as a pre-fermentation composition of 80 wt % Lactobacillus to 20 wt % fruit. The two components are mixed in an appropriate fermentation container. The temperature and pH of the mixture are maintained at appropriate conditions conducive to fermentation. Fermentation is allowed to proceed to completion. During fermentation, the Lactobacillus consumes sugars in the fruit and produces lactic acid. The Cocos Nucifera fruit includes lauric acid, a kind of medium chain triglyceride. The fermentation process is believed to make the lactic acid more bio-available.

Subsequent to fermentation, the fermented product is filtered to clarify the solution and remove any remaining feedstock material.

The finished antimicrobial product is suitable as an additive to personal care products and is effective to help retard or eliminate the growth of topical pathogens as well as to preserve the personal care products from spoiling or to substantially improve the shelf-life of a product. The antimicrobial product can also be used to preserve the function of various active ingredients including anti-oxidants (vitamins), emulsifiers, and surfactants. Testing has shown the antibacterial and antifungal effectiveness of the product as listed in Table 1, below.

TABLE 1
Minimum Inhibitory Concentration

| Organism | Minimum Inhibitory Concentration (wt %) |
|---|---|
| S. aureus | 2 |
| E. coli | 8 |
| P. aeruginosa | 8 |
| A. brasiliensis | 0.5 |
| C. albicans | 0.5 |

The finished product has an appearance that is clear to slightly hazy, with a color rated 5 maximum on the Gardner scale, and pH of 7.0 to 9.0. This makes the product suitable for inclusion in personal care products without introducing undesirable properties, such as off colors or odors.

Additional testing has demonstrated that the product is not a primary skin irritant or sensitizer, is not an eye irritant, is not harmful to aquatic organisms, and is not mutagenic to Salmonella or E. Coli.

The foregoing has described an antimicrobial composition and a method for its manufacture. All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends any novel one, or any novel combination, of the features disclosed in this specification (including any accompanying potential points of novelty, abstract and drawings), or to any novel one, or any novel combination, of the steps of any method or process so disclosed.

We claim:

1. An antimicrobial and antifungal composition, prepared by a process of using Lactobacillus to ferment Cocos Nucifera fruit.

2. The composition according to claim 1, wherein the Cocos Nucifera is macerated to produce a feedstock to be fermented by the Lactobacillus and create a fermented product.

3. The composition according to claim 2, wherein the proportions of feedstock to Lactobacillus in a pre-fermentation composition is selected to permit fermentation to proceed to completion.

4. The composition according to claim 2, wherein a pre-fermentation composition is about 20 wt % feedstock about 80 wt % Lactobacillus in a growth culture.

5. The composition according to claim 2, wherein the fermented product is filtered to clarify and remove remaining feedstock material and produce the composition.

6. An antimicrobial and antifungal composition for use with personal care products, prepared by a process of:
macerating white fruit of Cocos Nucifera to create a feedstock material;
mixing the feedstock material with a bacteria and using the bacteria to ferment the feedstock material to create a fermented product; and
filtering the fermented product to clarify the product and remove remaining feedstock material and produce the composition.

7. The composition according to claim 6, wherein the bacteria is Lactobacillus.

8. The composition according to claim 6, wherein a mass of the feedstock material is about one-quarter a mass of the bacteria to permit fermentation to proceed to completion.

9. The composition according to claim 6, wherein the composition has a pH of about 7.0 to about 9.0.

10. A method of making an antimicrobial and antifungal composition, comprising the steps of:
macerating white fruit of Cocos Nucifera to produce a feedstock material;
mixing the feedstock material with a bacteria to create a pre-fermentation composition and using the bacteria to ferment the feedstock material to create a fermented product; and
filtering the fermented product to remove remaining feedstock material and produce the composition.

11. The method according to claim 10, wherein a pre-fermentation composition is about 20 wt % feedstock about 80 wt % Lactobacillus in a growth culture to permit fermentation to proceed to completion.

12. The method according to claim 10, further including the step of maintaining a pre-selected temperature and a pre-selected pH to permit fermentation to complete.

13. The method according to claim 10, wherein the bacteria is Lactobacillus.

14. The method according to claim 10, wherein during fermentation, the bacteria consumes sugars in the feedstock material and produces lactic acid.

15. The method according to claim 10, wherein the composition has a pH of about 7.0 to about 9.0.
